(19) United States
(12) Patent Application Publication
(10) Pub. No.: US 2013/0078616 A1
(43) Pub. Date: Mar. 28, 2013

(54) METHOD AND SYSTEM FOR AMPLIFICATION OF NUCLEIC ACIDS IN MICROFLUIDIC VOLUME HYDROGELS

(71) Applicant: Alexey Atrazhev, Edmonton (CA)

(72) Inventor: Alexey Atrazhev, Edmonton (CA)

(73) Assignee: The Governors of the University of Alberta, Edmonton (CA)

(21) Appl. No.: 13/616,579

(22) Filed: Sep. 14, 2012

Related U.S. Application Data
(60) Provisional application No. 61/535,468, filed on Sep. 16, 2011.

(51) Int. Cl. C12M 1/34; G01N 21/75

(52) U.S. Cl. 435/5; 435/287.2; 435/288.7; 435/6.12; 435/6.11; 435/7.1; 436/501; 422/69

(57) ABSTRACT

The present invention provides for a novel method and system for amplification of nucleic acids within a hydrogel of microfluidic volume using a hydrocarbon wax as a support substrate.

### FIELD OF THE INVENTION

The present invention pertains to the field of amplification of nucleic acids in microlitre and nanolitre volumes.

### BACKGROUND OF THE INVENTION

All of the publications, patents and patent applications cited within this application are herein incorporated by reference in their entirety to the same extent as if the disclosure of each individual publication, patent application or patent was specifically and individually indicated to be incorporated by reference in its entirety.

### DESCRIPTION OF RELATED ART

The polymerase chain reaction (PCR) proceeds at temperatures close to the boiling point of water; therefore it requires constant protection against evaporation, in order to maintain constant concentrations of the reagents and buffer components within the reaction volume. In the case of conventional PCR performed in a thermocycler, and so long as both the tube and lid are heated, with a layer of mineral or paraffin oil placed over the reaction solution to provide a vapor barrier, a 300 μl polypropylene tube with a tight lid provides sufficient protection for reaction volumes greater than 10 μl. Paraffin wax (with longer chain alkanes than the oil and solid at room temperature) has also been used (Sparkman D. R., Gen Res (1992) 2:180-181), but the combination of tube, thermocycler and paraffin or mineral oil has become an industry standard. An alternative application of paraffin wax in PCR has been its use sequester important components of a PCR mixture that are liberated during pre-denaturation step and provide a “hot start” to the PCR reaction (Blair P. et al., Gen Res (1994) 4:191-194).

“On-chip” PCR in planar microfluidic chips is often performed with compartments enclosed with valves, which normally doesn't require oil (Zhang C. et al., Nucl Acids Res (2007) 35(13):4223-37). In an alternate scheme, nanolitre volumes of the PCR reaction mixture are injected, as droplets in mineral oil, into a microfluidic channel that passes repeatedly between the different heated zones required for the reaction (Kopp, M. U. et al., Science (1998) 280:1046-48; Schaerli Y. et al., Anal Chem (2009) 81(1) 302-306; Obeid, P. J. et al., Anal. Chem. (2003) 75, 288-295). In yet another alternative scheme, emulsion PCR, the reaction proceeds in picolitre droplets suspended in a tube with mineral oil (Williams R. et al., Nature Meth (2006) 3(7):545-550). Emulsion PCR excludes competition, allows all target molecules to amplify simultaneously and is practiced in amplification of complex cDNA libraries.

The preceding description of conventional and on-chip PCR refers to PCR in liquid-phase solution, which is still the predominant approach for deoxyribonucleic acid (DNA) amplification. An alternative medium supporting PCR is the cross-linked hydrogel covalently bound to glass. It has been exploited by Chetverin (U.S. Pat. No. 5,616,478) and Mitra (Mitra, R. D., Nucl Acid Res (1999) 27, 34e) for generating molecular colonies as well as by the inventors herein to make an open platform for molecular diagnostics (Atrazhev A. et al., Anal Chem. 2010; 82:8079-87). The utility of these hydrogel devices is significant, though there are drawbacks associated with the use of a mineral or paraffin oil layer in such a device. As such, there is a need in the present art for a device capable of performing nucleic acid amplifications without the use of a mineral oil or paraffin oil overlay.

### SUMMARY OF INVENTION

The present art is in need of hydrogel devices with reduced fragility, improved vapor barrier, increased stability during handling and transport and increased safety on disposal.

In one aspect, the present invention provides for a novel support substrate for isolating a multiplicity of hydrogel reaction chambers encapsulated within said support substrate with at most one surface of the hydrogel reaction chamber not in direct contact with said support substrate; said novel support substrate comprised of a hydrophobic material substantially solid at the temperature of non-operational handling or transport, wherein at temperatures relevant for monitoring of reactions within said hydrogel reaction chambers, said hydrophobic material becomes optically transparent at wavelengths relevant for the monitoring of said reactions undertaken in said hydrogel reaction chambers. In one embodiment said hydrophobic material is fluid at temperatures relevant for the monitoring of reaction undertaken in said hydrogel reaction chambers. In a further embodiment said hydrophobic material is optically transparent and non-fluorescing at wavelengths of 390-420 nm, and in an even more preferred embodiment, optically transparent and non-fluorescent at a wavelength of 405 nm.

In another aspect, the present invention provides for a novel support substrate for isolating a multiplicity of hydrogel reaction chambers encapsulated within said support substrate with at least one substantially planar surface not in direct contact with said support substrate and available for administration of a sample capable of interrogation by means of a reaction undertaken within said hydrogel reaction chamber; said novel support substrate comprised of a hydrophobic material substantially solid at the temperature of non-operational handling or transport, wherein at temperatures relevant for monitoring of reactions within said hydrogel reaction chambers, said hydrophobic material becomes optically transparent at wavelengths relevant for the monitoring of said reactions undertaken in said hydrogel reaction chambers. In one embodiment said hydrophobic material is fluid at temperatures relevant for the monitoring of the reaction undertaken in said hydrogel reaction chambers. In a further embodiment said hydrophobic material is optically transparent and non-fluorescing at wavelengths of 390-420 nm, and in an even more preferred embodiment, optically transparent and non-fluorescent at a wavelength of 405 nm. In one embodiment the reaction undertaken in said hydrogel reaction chamber is selected from the group comprised of reverse transcription, polymerase chain reaction, isothermal polymerase chain reaction, immunostaining, immunohistochemistry, immunolabeling, in situ hybridization, and viral lysis of eukaryotic and prokaryotic cells.

In another aspect the preset invention provides for a method of interrogating a sample for the presence or absence of a biological molecule by means of a reaction, comprising establishing at least one hydrogel reaction chamber containing a sampler within a support substrate, said hydrogel reaction chamber encapsulated within said support substrate with at most one surface of the hydrogel reaction chamber not in direct contact with said support substrate; said support substrate hydrophobic and substantially solid at the temperature of non-operational handling or transport, wherein at temperatures relevant for monitoring of reactions within said hydrogel reaction chambers, said support substrate becomes optically transparent at wavelengths relevant for the monitoring of said reactions undertaken in said hydrogel reaction chambers; initiating a reaction within said at least one hydrogel reaction chamber capable of producing an optical signal for monitoring of said reaction; and detecting the presence or absence of said optical signal. In one embodiment said support substrate is fluid at temperatures relevant for the monitoring of reaction undertaken in said hydrogel reaction chambers. In a further embodiment said support substrate is optically transparent and non-fluorescing at wavelengths of 390-420 nm, and in an even more preferred embodiment, optically transparent and non-fluorescent at a wavelength of 405 nm. In one embodiment the reaction undertaken in said hydrogel reaction chamber is selected from the group comprised of reverse transcription, polymerase chain reaction, isothermal polymerase chain reaction, immunostaining, immunohistochemistry, immunolabeling, in situ hybridization, and viral lysis of eukaryotic and prokaryotic cells.

In another aspect the preset invention provides for a method of interrogating a sample for the presence or absence of a biological molecule by means of a reaction, comprising establishing at least one hydrogel reaction chamber containing a sampler within a support substrate, said hydrogel reaction chamber encapsulated within said support substrate with at least one substantially planar surface not in direct contact with said support substrate and available for administration of a sample capable of interrogation by means of a reaction undertaken within said hydrogel reaction chamber; said support substrate hydrophobic and substantially solid at the temperature of non-operational handling or transport, wherein at temperatures relevant for monitoring of reactions within said hydrogel reaction chambers, said support substrate becomes optically transparent at wavelengths relevant for the monitoring of said reactions undertaken in said hydrogel reaction chambers; administering to said hydrogel reaction chamber a sample capable of interrogation by means of a reaction undertaken within said hydrogel reaction chamber; initiating a reaction within said at least one hydrogel reaction chamber capable of producing an optical signal for monitoring of said reaction; and detecting the presence or absence of said optical signal. In one embodiment said support substrate is fluid at temperatures relevant for the monitoring of reaction undertaken in said hydrogel reaction chambers. In a further embodiment said support substrate is optically transparent and non-fluorescing at wavelengths of 390-420 nm, and in an even more preferred embodiment, optically transparent and non-fluorescent at a wavelength of 405 nm. In one embodiment the reaction undertaken in said hydrogel reaction chamber is selected from the group comprised of reverse transcription, polymerase chain reaction, isothermal polymerase chain reaction, immunostaining, immunohistochemistry, immunolabeling, in situ hybridization, and viral lysis of eukaryotic and prokaryotic cells. In another embodiment the sample capable of interrogation by means of a reaction undertaken within the hydrogel reaction chamber is a human clinical sample. In a further embodiment, the human clinical sample is a tissue sample suspended in at least water. In a further embodiment, the human clinical sample is selected from the group comprised of blood, macerated tissue, lymphatic fluid, genital swab, nasopharyngeal swab, buccal swab, skin swab, bone marrow, saliva, urine, and fecal matter.

In one aspect, the present invention provides for a novel support substrate for isolating a multiplicity of hydrogel reaction chambers encapsulated within said support substrate with at most one surface of the hydrogel reaction chamber not in direct contact with said support substrate; said novel support substrate comprised of a hydrophobic material substantially solid at the temperature of non-operational handling or transport, wherein at temperatures relevant for monitoring of reactions within said hydrogel reaction chambers, said hydrophobic material is optically transparent at wavelengths relevant for the monitoring of said reactions undertaken in said hydrogel reaction chambers. In one embodiment said hydrophobic material is fluid at temperatures relevant for the monitoring of reaction undertaken in said hydrogel reaction chambers. In a further embodiment said hydrophobic material is optically transparent and non-fluorescing at wavelengths of 390-420 nm, and in an even more preferred embodiment, optically transparent and non-fluorescent at a wavelength of 405 nm.

In another aspect, the present invention provides for a novel support substrate for isolating a multiplicity of hydrogel reaction chambers encapsulated within said support substrate with at least one substantially planar surface not in direct contact with said support substrate and available for administration of a sample capable of interrogation by means of a reaction undertaken within said hydrogel reaction chamber; said novel support substrate comprised of a hydrophobic material substantially solid at the temperature of non-operational handling or transport, wherein at temperatures relevant for monitoring of reactions within said hydrogel reaction chambers, said hydrophobic material is optically transparent at wavelengths relevant for the monitoring of said reactions undertaken in said hydrogel reaction chambers. In one embodiment said hydrophobic material is fluid at temperatures relevant for the monitoring of the reaction undertaken in said hydrogel reaction chambers. In a further embodiment said hydrophobic material is optically transparent and non-fluorescing at wavelengths of 390-420 nm, and in an even more preferred embodiment, optically transparent and non-fluorescent at a wavelength of 405 nm. In one embodiment the reaction undertaken in said hydrogel reaction chamber is selected from the group comprised of reverse transcription, polymerase chain reaction, isothermal polymerase chain reaction, immunostaining, immunohistochemistry, immunolabeling, in situ hybridization, and viral lysis of eukaryotic and prokaryotic cells.

In another aspect the preset invention provides for a method of interrogating a sample for the presence or absence of a biological molecule by means of a reaction, comprising establishing at least one hydrogel reaction chamber containing a sampler within a support substrate, said hydrogel reaction chamber encapsulated within said support substrate with at most one surface of the hydrogel reaction chamber not in direct contact with said support substrate; said support substrate hydrophobic and substantially solid at the temperature of non-operational handling or transport, wherein said support substrate is optically transparent at wavelengths relevant for the monitoring of said reactions undertaken in said hydrogel reaction chambers; initiating a reaction within said at least one hydrogel reaction chamber capable of producing an optical signal for monitoring of said reaction; and detecting the presence or absence of said optical signal. In one embodiment said support substrate is fluid at temperatures relevant for the monitoring of reaction undertaken in said hydrogel reaction chambers. In a further embodiment said support substrate is optically transparent and non-fluorescing at wavelengths of 390-420 nm, and in an even more preferred embodiment, optically transparent and non-fluorescent at a wavelength of 405 nm. In one embodiment the reaction undertaken in said hydrogel reaction chamber is selected from the group comprised of reverse transcription, polymerase chain reaction, isothermal polymerase chain reaction, immunostaining, immunohistochemistry, immunolabeling, in situ hybridization, and viral lysis of eukaryotic and prokaryotic cells.

In another aspect the preset invention provides for a method of interrogating a sample for the presence or absence of a biological molecule by means of a reaction, comprising establishing at least one hydrogel reaction chamber containing a sampler within a support substrate, said hydrogel reaction chamber encapsulated within said support substrate with at least one substantially planar surface not in direct contact with said support substrate and available for administration of a sample capable of interrogation by means of a reaction undertaken within said hydrogel reaction chamber; said support substrate hydrophobic and substantially solid at the temperature of non-operational handling or transport, wherein said support substrate is optically transparent at wavelengths relevant for the monitoring of said reactions undertaken in said hydrogel reaction chambers; administering to said hydrogel reaction chamber a sample capable of interrogation by means of a reaction undertaken within said hydrogel reaction chamber; initiating a reaction within said at least one hydrogel reaction chamber capable of producing an optical signal for monitoring of said reaction; and detecting the presence or absence of said optical signal. In one embodiment said support substrate is fluid at temperatures relevant for the monitoring of reaction undertaken in said hydrogel reaction chambers. In a further embodiment said support substrate is optically transparent and non-fluorescing at wavelengths of 390-420 nm, and in an even more preferred embodiment, optically transparent and non-fluorescent at a wavelength of 405 nm. In one embodiment the reaction undertaken in said hydrogel reaction chamber is selected from the group comprised of reverse transcription, polymerase chain reaction, isothermal polymerase chain reaction, immunostaining, immunohistochemistry, immunolabeling, in situ hybridization, and viral lysis of eukaryotic and prokaryotic cells. In another embodiment the sample capable of interrogation by means of a reaction undertaken within the hydrogel reaction chamber is a human clinical sample. In a further embodiment, the human clinical sample is a tissue sample suspended in at least water. In a further embodiment, the human clinical sample is selected from the group comprised of blood, macerated tissue, lymphatic fluid, genital swab, nasopharyngeal swab, buccal swab, skin swab, bone marrow, saliva, urine, and fecal matter.

### BRIEF DESCRIPTION OF THE FIGURES

FIG. 1 shows a schematic of the instrument capable of performing PCR and Melting Curve Analysis (MCA) as contemplated herein;

FIG. 2 shows a schematic of the loading of nucleic acids and running PCR in hydrogel post arrays immersed in wax;

FIG. 3 shows the manufacturing of wax-covered hydrogel post arrays;

FIG. 4 shows a cross-sectional schematic of hydrogel polymerized in the wells of the mould;

FIG. 5 shows a cross-sectional schematic of the open polymerizing of hydrogel post array in nitrogen following immersion in wax;

FIG. 6 shows the distribution of DNA binding ChargeSwitch™ beads to hydrogel posts in association with a cross-sectional schematic of the relevant device;

FIG. 7 shows PCR amplification of a 100 b.p. fragment from BKV genome in a hydrogel post array in wax;

FIG. 8 shows PCR amplification from the HSV-2 genome in a hydrogel post array in wax; and

FIG. 9 shows MCA analysis of P. falciparum dhps PCR product in hydrogel post arrays in oil (a,c) and in wax (b,d).

### DETAILED DESCRIPTION OF THE PRESENT INVENTION

There are obvious issues with liquid oil as a vapour barrier during nucleic acid detection, PCR amplification or transport of the device.
It needs to be confined inside the pan, otherwise it spills over the working area;
Coverage of the reaction area with a lid causes condensation and hinders optical detection;
Loading the DNA sample evenly to the posts under oil is inconvenient because aqueous sample has to be driven through hydrophobic oil before it reaches the surface of the post;
It is difficult to deliver the sample in equal manner to all posts under these conditions; and
The pan needs to be rigid and therefore is machined out of metal, which is expensive, so there is pressure to use the pan repeatedly which can cause cross-contamination.
   Therefore the art is in need of an improved way to provide a vapour barrier for use with hydrogel posts in a device as contemplated herein.

The present invention contemplates use of any hydrophobic material or substance such as: 1) straight or branched hydrocarbon, with potentially some or all hydrogen atoms replaced with halogen, chalcogen or pnicogen atoms; 2) solid (poly) ethers and (poly)esters; 3) aliphatic or aromatic acids. In an additional aspect, the invention contemplates the vapor barrier may be fluid or may be comprised of a solid air-tight support such as a plastic. An ideal material that undergoes liquefaction at temperatures relevant for the monitoring of the reaction undertaken within the hydrogel, as contemplated by the present invention, is hydrophobic; exhibits limited fluorescence under long-wave UV or visible excitation source in liquid state; exhibits optical transparency in the liquid state (though translucence may suffice); has a melting point higher than, at a minimum, room temperature, though in a preferred embodiment, above 50° C.; and has a melting and freezing point below the lowest temperature used within the PCR reaction. In a preferred embodiment, the material would show melting point hysteresis, with a melting point of 70° C. and a freezing point below the lowest PCR temperature. Due to the choices of melting point, freezing point and optical transparency in the liquid state, the hydrocarbon contemplated by the present invention does not interfere with image acquisition and stays liquid during PCR cycling. To facilitate optical detection, a material that has maximum transparency at temperatures relevant for the monitoring of the reaction undertaken within the hydrogel is preferred. Advantageously, when this type of vapour barrier solidifies after completion of thermocycling or isothermal amplification, it allows for safe disposal of materials representing a potential biohazard used as template for the amplification (for example clinical samples).

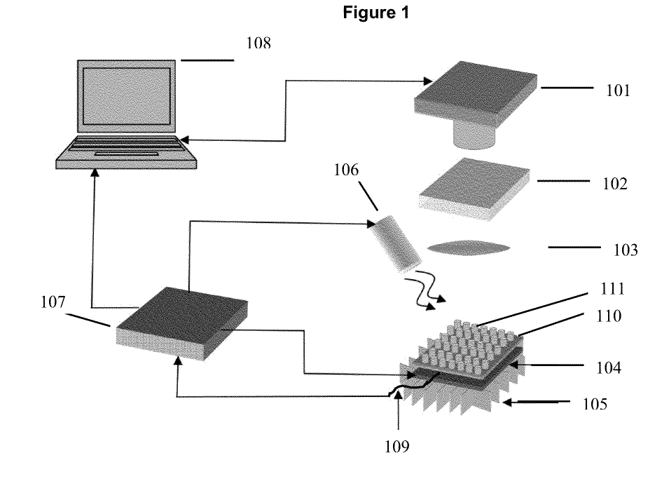

The nucleic acid amplification and detection system of the prior art is an array of hydrogel posts bound to a thin glass wafer, placed in an aluminum pan and immersed in mineral oil to prevent evaporation, more fully described in Atrazhev A. et al. (Atrazhev A. et al., Anal Chem. 2010; 82:8079-87), the system for which is shown in FIG. 1. Each hydrogel post arrayed on thin glass 110 of approximate 0.6 μl volume, with all PCR reagents present and functions as an independent reaction vessel. The glass containing the hydrogel post array is placed on a thermal device 104, such as a Peltier thermoelectric device, for thermal cycling, with optional heatsink 105 placed in thermal communication with the Peltier device. Temperature sensor 109 is placed in thermal communication with heatsink 105, or absent the optional heatsink thermal device 104 (not shown) and gel post array 110. Detection is carried out by CCD camera 101 through mineral oil that covers the posts (FIG. 1), with an optical interference filter (510 nm centre; 50 nm bandwidth) 102 and biconvex lens (f=25.4 mm) 103 interposed between the CCD camera 101 and hydrogel posts 111, forming the hydrogel post array. Temperature, as measured by temperature sensor 109, is recorded and controlled by microprocessor 107, which also controls diode laser (405 nm, 65 mW) 106 that illuminates hydrogel array 110. Computer 108 is in electronic communication with controlling microprocessor 107 and CCD camera 101 to provide controlling and operating instructions to the devices and to further receive images and temperature data respectively:

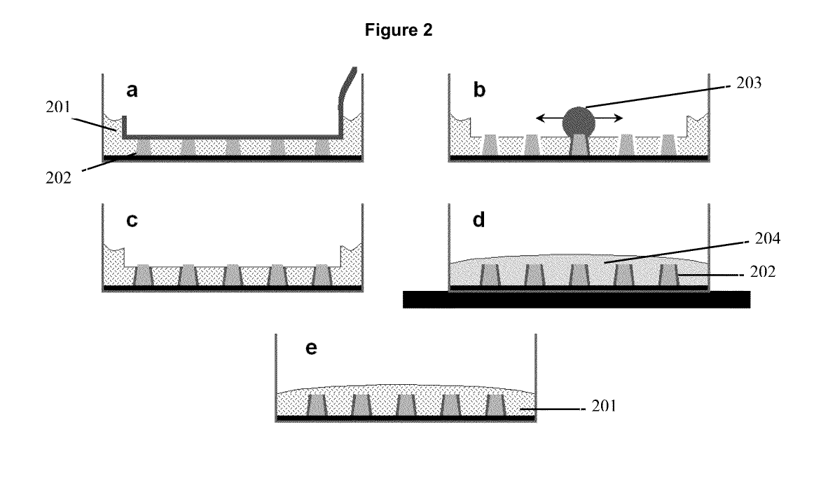

To solve the limitations and problems of the prior art, the present invention contemplates a gel post array embedded in a wax 201 instead of mineral oil (FIG. 2(a)) such as, in its preferred embodiment, paraffin wax. At room temperature, the wax-based framework for the hydrogel posts 202 is solid and the wax provides a substantially planar generally hydrophobic surface that dramatically facilitates transfer of water based sample 203 to the hydrophilic hydrogel posts 202 (FIG. 2(b), FIG. 2(c)) interrupting the wax surface. As a further benefit, the hardness of the wax allows the pan be made of foil and therefore glass plate 110 as shown in FIG. 1 may optionally be replaced with foil, for reduced cost or disposability, with thermal device 104 or optional heatsink 105 providing a solid support for the wax entombed hydrogel post array while the wax is in a molten state. During the PCR cycle the wax melts into molten form 204, and flows over and around thereby protecting hydrogel posts 202 from drying, in a similar fashion as mineral oil (FIG. 2(d)). After the PCR/MCA cycle is completed, the wax solidifies 201 and makes discarding of biohazardous samples much safer (FIG. 2(e)). As will be obvious to one skilled in the art, the wax-based post arrays have numerous benefits, which include but are not limited to, ease of manufacture while still being useable within the instruments of the art as depicted in FIG. 1.

It is contemplated by the present invention that the framework described herein can advantageously be used for hydrogels within which DNA amplification or other nucleic acid amplification can be undertaken, or within which other reactions benefiting from isolation within an array, including but not limited to reverse transcription PCR, isothermal PCR, cellular immunoassays, or in situ hybridization. Further, various configurations of hydrogel, partially encapsulated or otherwise, are contemplated as benefitting from the wax framework of the present invention; including hydrogel strips, hydrogels partially encapsulated by a capillary or hydrogel “spots”, or other hydrogel formations or formulations as known in the art.

As used herein, wax means a mixture of saturated and/or unsaturated hydrocarbons with melting points greater than room temperature or the temperature generally experienced in the handling of the manufactured hydrogel post array. Paraffin wax is defined as a mixture of solid saturated hydrocarbons with melting point of 45-66° C. (Table 1). It is contemplated that the optimal wax for use in gel post array is chosen based on its demonstrating minimal fluorescence in the liquid state under applicable laser illumination, in the preferred embodiment 405 nm, with several waxes listed in Table 2. ParaPlast™ embedding wax manufactured by Leica Co. for biopsies was selected.

TABLE 1
n-Alkanes (straight saturated hydrocarbons) melting
between 44° C. and 81° C. (from Handbook
of Chemistry and Physics, 45th Ed., section C).

| | | Melting | Density |
|---|---|---|---|
| Name | Formula | point ° C. | (liquid) |
| n-Docosane | CH₃(CH₂)₂₀CH₃ | 44.4 | 0.7778 |
| n-Tricosane | CH₃(CH₂)₂₁CH₃ | 47.4 | 0.7797 |
| n-Tetracosane | CH₃(CH₂)₂₂CH₃ | 51.1 | 0.7786 |
| n-Pentacosane | CH₃(CH₂)₂₃CH₃ | 53.3 | |
| n-Triacontane | CH₃(CH₂)₂₈CH₃ | 65.5 | 0.7750 |
| n-Pentatriacontane | CH₃(CH₂)₃₂CH₃ | 74.6 | 0.7814 |
| n-Tetracontane | CH₃(CH₂)₃₈CH₃ | 81.0 | |

TABLE 2
Different waxes tested with gel posts.

| | Melting point | Fluorescence |
|---|---|---|
| Wax brand | (° C.) | at 405 nm |
| IKEA tea-lights | 58 | High |
| NeoWax ™ by Dentsply Trubyte | 64 | very high |
| BDH Paraffin wax | 49 | Low |
| Leica ParaPlast ™ | 52 | very low |

The present invention contemplates the wax entombed hydrogel post arrays as being placed face-up, with the posts resting on or within depressions on the glass plate or mould in optical communication with, and in its preferred embodiment adjacent to, the optical detection components, as shown in FIG. 1. It is also contemplated that the hydrogel post array can be face-down during the course of the PCR, with a substrate optically transparent to the fluorescence and excitation wavelengths interposed between the hydrogel posts and illumination source and optical receiving device (for example in the prior art device contemplated herein and described in FIG. 1, diode laser 106 and CCD camera 101, respectively). To prepare these inverted hydrogel post arrays, first a wax frame is made by placing a stamp similar to that of described in Example 1 herein (having the same x-y footprint as the glass plate or mould holding the posts, but no surface seal) into an aluminum foil pan on a heating element, pouring molten wax around the stamp, and cooling the assembly to room temperature. The stamp is then removed to provide a wax frame in the aluminum pan into which the gel array on glass can be inserted, inverted such that the hydrogel post array resides within the cavity described by the wax frame. The sample is applied by one of two methods: 1) aliquoting sample into the rectangular cavity of the wax frame followed by insertion of the hydrogel post array within the cavity described by the wax frame, allowing the sample to be absorbed into the posts, or 2) aliquoting sample to the surface of the posts, adding a small amount of water to the wax frame cavity for hydration purposes, and inserting the hydrogel post array within the cavity described by the wax frame. With either method, the posts may be briefly air-dried before sample application to enhance sample uptake in the posts. Following sample application, with the gel post array inverted within the wax frame cavity, the assembly is placed on the instrument's thermal device, as shown in FIG. 1, and thermocycling is begun to perform PCR and melt the wax.

While glass is normally used in all embodiments for the plate or mould that supports the hydrogel, other transparent or opaque materials may be appropriate, including various soft polymeric materials (e.g. polydimethyl siloxane (PDMS) and other silicones), hard polymeric materials (e.g. poly(methylmethacrylate), cyclic olefin polymers and copolymers), metals (e.g. copper, anodized aluminum) or ceramic materials. Transparent materials are necessary to perform the fluorescence detection, but pans, plates and other components as may be conceived, not in the optical path, may be opaque. Use of materials reflective of the fluorescence optical signal, in conjunction with a multiplicity of hydrogel posts within an array, is not advised, due to the confusion of fluorescence signals. Care and consideration should be paid to the thermal transference properties of the material to ensure consistent and rapid temperature changes throughout the hydrogel posts during PCR. This is particularly relevant for materials interposed between the thermal device and the hydrogel posts.

Storage of hydrogel with reagents can be accomplished by refrigeration, for short-term storage, or by a desiccation-rehydration procedure, for long-term storage. The short-term refrigeration storage procedure entails placing the wax- and surface seal-enclosed post array in a refrigerator (4° C.) for the period desired, prior to removing the covering surface seal, as described herein.

The long-term desiccation—rehydration procedure is as follows. Desiccation begins with removing the surface seal cover and contemporaneously dehydrating the hydrogel under controlled desiccation conditions using, for example, a desiccation chamber. Upon removal from the desiccation chamber, a new seal is established by immediately placing the post array on a heating element, re-introducing the stamp with a new surface seal, allowing the wax to melt and seal against it, and removing the stamp to leave wax- and surface-sealed desiccated posts. It is contemplated by the present invention that rehydration occurs immediately preceding use, either with water, followed by application of sample for PCR interrogation, or by sample dissolved or suspended in water. Rehydration entails removing the surface seal and immediately applying water or sample to the post arrays, in a preferred embodiment, by flooding the entire array of hydrogel posts or other types of hydrogel reaction unit simultaneously with the sample or alternatively by adding sample to each post individually. In the process of rehydration, sample is absorbed into the hydrogel as it flows into the hydrogel in the wax through the openings exposed by removal of the sealing material. The sample may be a raw sample (e.g. urine, swabs, blood, sputum), purified DNA or other nucleic acid, or DNA or other nucleic acid anchored on beads. Once sample application has occurred, the hydrogel is placed on the instrument's thermal device, and template amplification occurs with concomitant or subsequent product detection.

Hydrogels mounted on the heating element during wax entombing or otherwise during preparation of the device contemplated by the present invention and as described herein, already have the primers and thermostable DNA polymerase that can become activated and form primer-dimers. To prevent this, the magnesium precipitate hot start method can be used (Barnes W M. et al., Molecular and Cellular Probes (2002) 16:167-171), although it is contemplated that the present invention can be used either with or without a hot start step. In the magnesium hot start method, magnesium is sequestered as solid magnesium hydrogen phosphate precipitate (MgHPO₄(s)) dispersed in the PCR reagent solutions and later gel posts; it dissolves at 94° C. in the initial denaturation step and stays dissolved during all steps of the PCR cycle. Since DNA polymerases are inactive without magnesium, the amplification initiates when the primer-dimer dissociates and the primers can bind their specific targets on genomic DNA.

The present invention demonstrates that replacing oil with wax has no significant adverse effect on nucleic acid amplification and analysis parameters already achieved for the gel post platform or other geometries, forms or encapsulations of hydrogels, including capillary-encased hydrogel, hydrogel strips or other forms, incorporating all reagents necessary for undertaking a reaction; all reagents necessary for undertaking a reaction on a yet to be added sample which may or may not include a biological molecule, for interrogation (by way of non-limiting example, a template nucleotide); or only a portion of the reagents necessary for undertaking a reaction, the remainder added in conjunction with the yet to be added sample which may or may not include a biological molecule. The present invention greatly improves the convenience of integrating the gel post array into a final point-of-care diagnostic device. After PCR/MCA completion, the wax solidifies, isolating the posts with clinical sample on them and can safely be discarded.

### EXAMPLE 1

### Wax Entombed Hydrogel Post Array

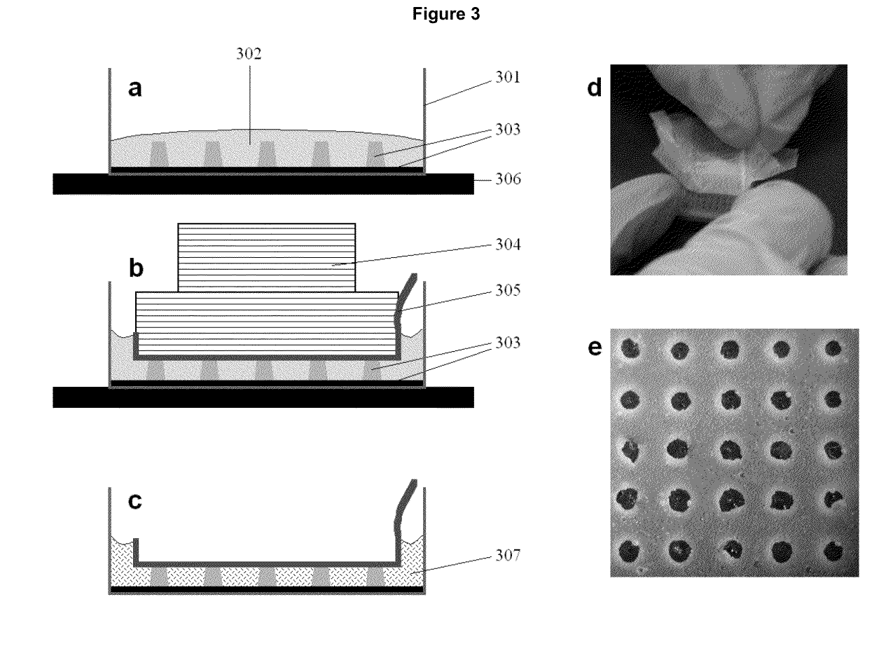

Fabrication of wax-filled gel post arrays takes place on a heating element set at 58° C. (FIG. 3(a)(b)(c)). First a disposable pan is made by folding aluminum foil 301 around a metal jig (not shown). The pan is then mounted on heating element 306, the hydrogel array 303, prepared separately and by means known in the art (see, by way of non-limiting example, WO2012027832), is placed inside and molten wax 302 is poured over sufficient to cover the posts in a manner similar to mineral oil as known in the prior art (FIG. 3(a)). Stamp 304 covered with a surface seal 305, such as Parafilm™, is placed on top of the post array 303 (FIG. 3(b)), making contact with the posts; following which the whole assembly is allowed to cool, or optionally cooled, to room temperature. Stamp 304, used for sealing the surface has an outer layer of PDMS silicone resin or other product so that the stamp easily detaches from surface seal 305 and leaves the posts covered with the surface seal. This surface seal covered stamp (coated with PDMS silicone resin to facilitate detachment) is hereafter termed a surface sealant. Materials other than Parafilm™ are also envisioned for the surface seal, functioning to exclude wax from the tops of the gel posts. A barrier formed from excess solid wax 307 is produced around the planar pit, and the tops of the posts lie on a substantially planar surface just beneath the surface seal 305 (FIG. 3(c)). When surface seal 305 is removed (FIG. 3(d) and FIG. 3(e)), the sample is preferably immediately applied and processed as previously described (Atrazhev A. et al., Anal Chem. 2010; 82:8079-87) with the exemplar device shown in FIG. 1, as the posts are exposed to the air and prone to evaporation. After the wax melts during processing as previously described it flows over the posts and covers them, preventing evaporation.

### EXAMPLE 2

### Glass Mould Hydrogel Array

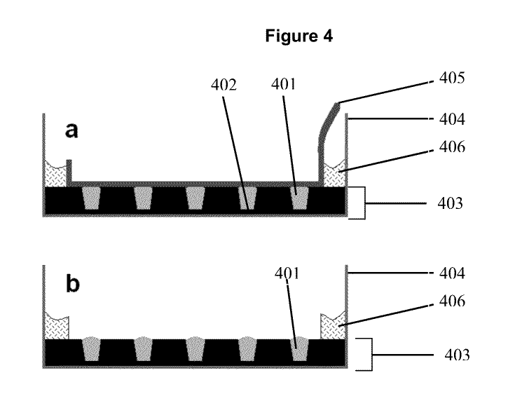

The hydrogel post array may be formed using a glass mould, allowing the hydrogel array to remain in the mould and not require transfer to a glass plate. It is also contemplated that a rigid mould may be used, by way of non-limiting example, a metal, such as aluminum, mould as previously described, or a polymer, such as plastic mould, as will be obvious to one skilled in the art. As shown in FIG. 4(a), the hydrogel posts 401 contained in each mould cavity 402 are thus enclosed by the glass mould 403 on all sides except their top surfaces, which are flush with (or may protrude slightly from) the top surface of the mould, and are open at the top. Then, as in Example 1, the hydrogel post array (now enclosed in a glass mould) is placed inside the aluminum foil pan 404 on a heating element(not shown), molten wax is poured on top to cover the posts, a stamp with a surface sealant is placed on top of the post array (not shown), and the whole assembly is cooled to room temperature, leaving a barrier formed from excess wax 406. The stamp is removed, leaving the posts covered with the surface seal 405. When sample analysis is required, the surface seal is removed as in FIG. 4(b) and the sample is applied immediately and processed as for Example 1.

### EXAMPLE 3

### Wax Patterning Technique

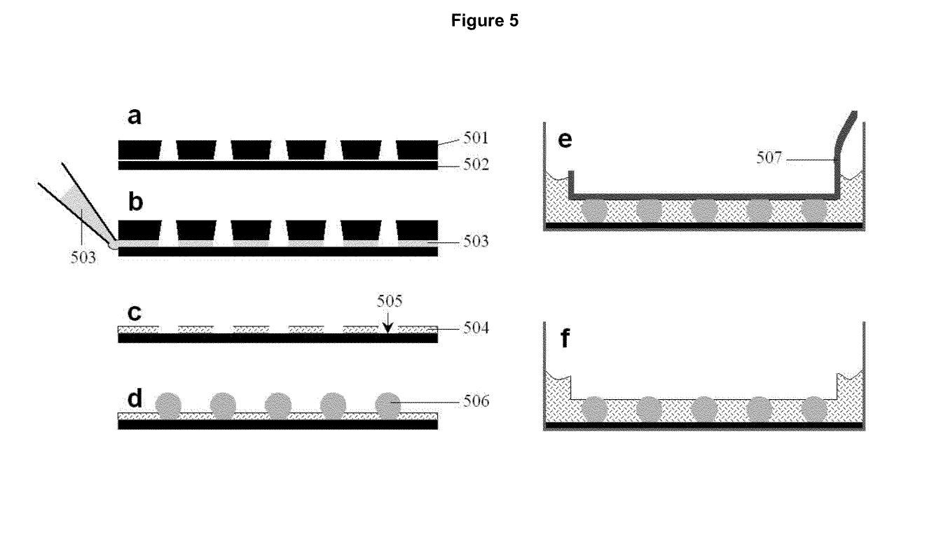

Alternatively, the gel post array is made directly on the glass plate to be inserted into the pan via a wax patterning technique, with no need for separate post fabrication (as in Examples 1 and 2) nor transfer to a separate plate (as in embodiment 1). As shown in FIG. 5(a), FIG. 5(b) and FIG. 5(c), first a patterned wax template is formed. A glass template 501 with an array of drilled through-holes identical to the cavities in the mould is placed on (above and in contact with) a glass slide 502; the glass assembly is transferred to and heated on a heating element and molten wax 503 is introduced into the assembly as shown in FIG. 5(b). The wax wicks in between the two glass plates covering the entire area where they touch, but it is absent in the areas in the lower glass slide that lie beneath holes in the upper glass template. The two pieces of glass are separated in this process; absent positive pressure to introduce the wax, relying entirely on the wicking through the contact area between the two pieces of glass, it will generate a wax layer on the order of 10-40 μm in thickness, varying on the wax, temperature of the molten wax used and the ambient air pressure. The two plates are then cooled, allowing the wax to solidify, separated (e.g. with a scalpel), and the top glass template is removed. The glass slide supports a patterned solid wax layer 504 with an array of circular bare glass spots 505 as shown in FIG. 5(c). Other spot geometries are also contemplated.

Next, the plate is moved to an oxygen-free atmosphere (e.g. nitrogen glove box or equivalent), a primer mixture is applied to the bare glass spots as appropriate for the assay in question, the mixture containing all reagents required for hydrogel polymerization as well as for PCR with the exception of the template DNA to be provided later with the sample. These liquid mixtures form isolated drops 506 on the bare glass spots separated from each other by the wax pattern as shown in FIG. 5(d). The gel in each droplet is allowed to polymerize as described previously by Atrazhev et al. Each polymerized droplet-shaped post in this array is bound at its base by the circular bare glass area in the patterned wax, and will range in shape depending on the size of the bare glass spot and the volume of reagents added; a variety of shapes are contemplated. Once the posts are fully polymerized, the array of posts on the glass plate is then treated in the same manner as that in Example 1 for entombment in wax with a surface seal 507 as shown in FIG. 5(e) and subsequent exposure of the post tops for sample application as shown in FIG. 5(f).

### EXAMPLE 4

### PCR Following Distribution of DNA Binding Beads

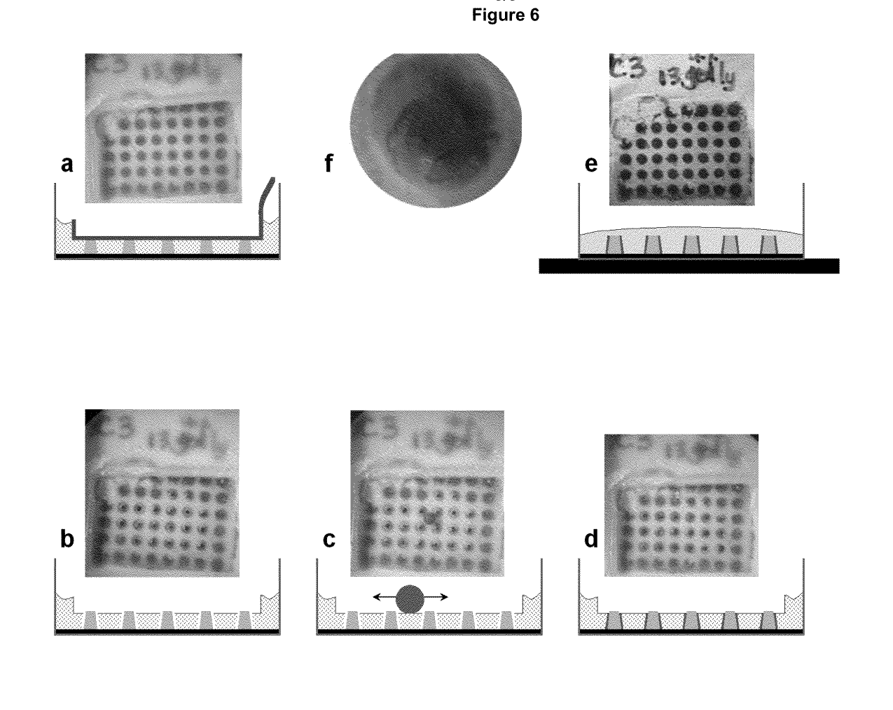

Using the system of the present invention and as shown in FIG. 6 by photographs of the device with schematic cross-sectional drawings underneath representing the state of the device, uniform distribution of DNA binding beads among the gel posts was observed and PCR/MCA data was generated, demonstrating equivalence of the wax-based and oil-based systems. The hydrogel posts were stained with Bromophenol Blue for better visibility. FIG. 6(a) shows a hydrogel post array chip covered with a Parafilm™ surface seal; FIG. 6(b) shows an uncovered chip with the surface seal removed; FIG. 6(c) shows the chip moments after it was uncovered in FIG. 6(b) with a 3 μl drop of a ChargeSwitch™ bead suspension deposited at the centre of the array (and with the posts having air-dried and shrunk somewhat in that time); FIG. 6(d) shows the chip after the drop has been manipulated throughout the uncovered wax surface, coming into contact with, and infusing, the bead suspension into each exposed hydrogel post's surface, rehydrating the post in the process; FIG. 6(e) shows the chip after heating results in the melting of the wax covering the bead-infused posts; and FIG. 6(f) shows the bead distribution in a representative hydrogel post as seen through a binocular microscope. A similar distribution of template is observed for raw sample, purified DNA, partially processed DNA, other nucleic acids or other materials linked to or containing template sequences.

### EXAMPLE 5

### Target Amplification from Viral DNA

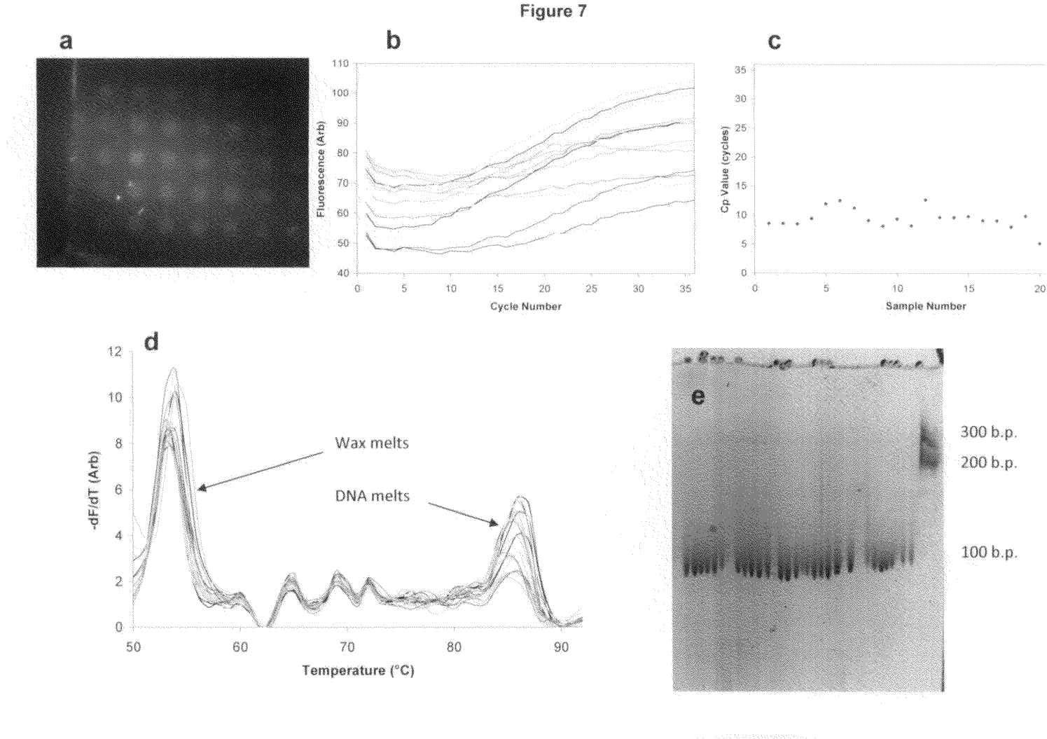

Specific targets from genomic DNA were successfully amplified by PCR using primers designed to selectively amplify pathogenic virus nucleic acid such as BK virus (BKV) and herpes simplex 2. FIG. 7 shows amplification of BKV template using the system and method of the present invention. FIG. 7(a) shows an image of a post array following 45 PCR cycles using BKV viral nucleic acid as a template, under conditions and with a system as described by Atrazhev et al. (Atrazhev A. et al., Anal Chem. 2010; 82:8079-87) with fluorescence confined to the hydrogel posts indicating increased presence of template polynucleotide DNA. FIG. 7(b) shows fluorescence (y-axis) as related to thermal cycle number (x-axis) for the BKV PCR reaction. FIG. 7(c) shows the Cₚ value for each PCR reaction, where Cₚ is defined as the PCR cycle at which the second derivative of the fluorescence curve for a given PCR reaction volume (i.e. a hydrogel post) is at a maximum. FIG. 7(d) shows a melting curve analysis (MCA) graph of individual hydrogel posts demonstrating substantial redundancy for the reactions within a uniform hydrogel post array, while FIG. 7(e) shows a polyacrylamide gel electrophoresis separation of individual posts.

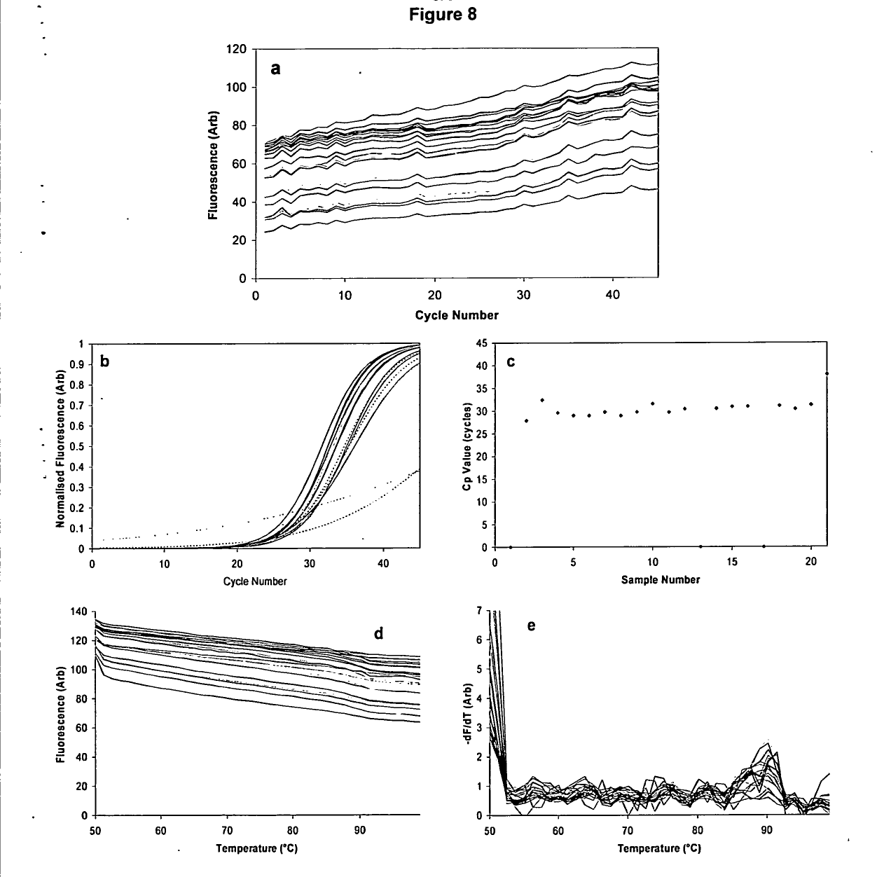

FIG. 8 shows amplification of herpes simplex 2 using the system and method of the present invention, using as template an unprocessed genital swab. FIG. 8(a) shows the raw fluorescence data (y-axis) versus thermal cycle number (x-axis), while FIG. 8(b) shows the normalized data from quantification of the preceding fluorescence data. FIG. 8(c) shows the Cₚ values, as defined above. FIG. 8(d) shows the fluorescence drop during DNA melting within hydrogel posts (melting curve), while FIG. 8(e) shows the MCA traces (first derivative of melting curves in FIG. 8(d)) for individual hydrogel posts. All instrumentation sets, reaction conditions and data processing are as described previously by Atrazhev et al. (Atrazhev A. et al., Anal Chem. 2010; 82:8079-87).

### EXAMPLE 7

### Analysis of Pre-Amplified DNA in Wax and Mineral Oil Protected Systems

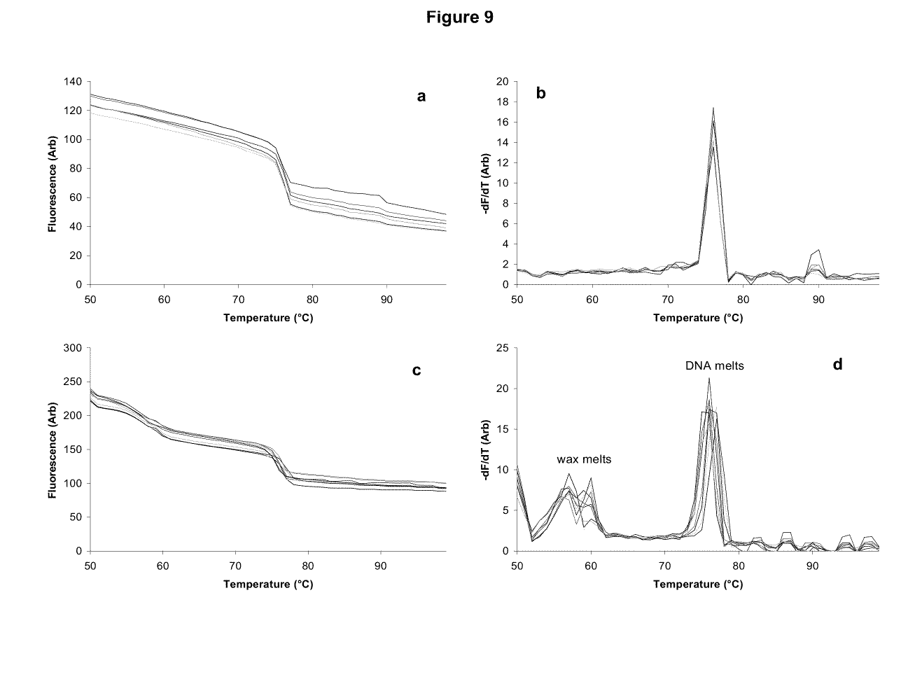

FIG. 9 shows the melting of pre-amplified Plasmodium falciparum DNA was also comparatively analyzed in gel posts immersed in wax as well as in mineral oil. FIGS. 9(a) and (b) show melting curves and their first derivative analyses, respectively, for P. falciparum dhfr gene PCR product in gel posts in oil, while FIGS. 9(c) and (d) show the same in wax. FIG. 9(a) and FIG. 9(c) show the fluorescence drop with temperature (melting curves) for oil and wax as a vapour barrier, respectively. FIG. 9(b) and FIG. 9(d) show the MCA graphs (first derivative of melting curves) for oil and wax as a vapour barrier, respectively. Note that, in FIGS. 9(c) and (d), there are two melting events arising from both the wax and DNA melting, and the associated drops in fluorescence. All instrumentation sets and reaction conditions were used as described previously by Atrazhev et al. (Atrazhev A. et al., Anal Chem. 2010; 82:8079-87). This demonstrates the equivalence of the two vapour barrier media for signal readout and temperature resolution.

While particular embodiments of the present invention have been described in the foregoing, it is to be understood that other embodiments are possible within the scope of the invention and are intended to be included herein. It will be clear to any person skilled in the art that modifications of and adjustments to this invention, not shown, are possible without departing from the spirit of the invention as demonstrated through the exemplary embodiments. The invention is therefore to be considered limited solely by the scope of the appended claims.

What is claimed is:

1. A support substrate for isolating a multiplicity of hydrogel reaction chambers encapsulated within said support substrate, with at most one surface of the hydrogel reaction chamber not in direct contact with said support substrate; said novel support substrate comprised of a hydrophobic material substantially solid at the temperature of non-operational handling or transport, wherein at temperatures relevant for monitoring of reactions within said hydrogel reaction chambers, said hydrophobic material becomes optically transparent at wavelengths relevant for the monitoring of said reactions undertaken in said hydrogel reaction chambers.

2. The support substrate of claim 1 wherein said hydrophobic material is fluid at temperatures relevant for the monitoring of reaction undertaken in said hydrogel reaction chambers.

3. The support substrate of claim 2 wherein said hydrophobic material is optically transparent and non-fluorescing at wavelengths of 390-420 nm.

4. The support substrate of claim 3 wherein said hydrophobic material is optically transparent and non-fluorescent at a wavelength of 405 nm.

5. The support substrate of claim 4 wherein said hydrophobic material is paraffin.

6. A support substrate for isolating a multiplicity of hydrogel reaction chambers encapsulated within said support substrate with at least one substantially planar surface not in direct contact with said support substrate and available for administration of a sample capable of interrogation by means of a reaction undertaken within said hydrogel reaction chamber; said novel support substrate comprised of a hydrophobic material substantially solid at the temperature of non-operational handling or transport, wherein at temperatures relevant for monitoring of reactions within said hydrogel reaction chambers, said hydrophobic material becoming optically transparent at wavelengths relevant for the monitoring of said reactions undertaken in said hydrogel reaction chambers.

7. The support substrate of claim 6 wherein said hydrophobic material is fluid at temperatures relevant for the monitoring of reaction undertaken in said hydrogel reaction chambers.

8. The support substrate of claim 7 wherein said hydrophobic material is optically transparent and non-fluorescing at wavelengths of 390-420 nm.

9. The support substrate of claim 8 wherein said hydrophobic material is optically transparent and non-fluorescent at a wavelength of 405 nm.

10. The support substrate of claim 8 wherein said hydrophobic material is paraffin.

11. A method of interrogating a sample for the presence or absence of a biological molecule by means of a reaction, comprising
(a) Establishing at least one hydrogel reaction chamber containing a sample within a support substrate,
said hydrogel reaction chamber encapsulated within said support substrate with at most one surface of the hydrogel reaction chamber not in direct contact with said support substrate;
said support substrate hydrophobic and substantially solid at the temperature of non-operational handling or transport,
wherein at temperatures relevant for monitoring of reactions within said hydrogel reaction chambers, said support substrate becomes optically transparent at wavelengths relevant for the monitoring of said reactions undertaken in said hydrogel reaction chambers;
(b) Initiating a reaction within said at least one hydrogel reaction chamber capable of producing an optical signal indicating the presence or absence of a biological molecule; and
(c) Detecting the presence or absence of said optical signal.

12. The method of claim 11 wherein said hydrophobic material is fluid at temperatures relevant for the monitoring of reaction undertaken in said hydrogel reaction chambers.

13. The method of claim 12 wherein said hydrophobic material is optically transparent and non-fluorescing at wavelengths of 390-420 nm.

14. The method of claim 13 wherein said hydrophobic material is optically transparent and non-fluorescent at a wavelength of 405 nm.

15. The method of claim 14 wherein said hydrophobic material is paraffin.

16. The method of claim 11 wherein the reaction undertaken in said hydrogel reaction chamber is selected from the group comprised of reverse transcription, polymerase chain reaction, isothermal polymerase chain reaction, immunostaining, immunohistochemistry, immunolabeling, in situ hybridization, and viral lysis of eukaryotic and prokaryotic cells.

17. A method of interrogating a sample for the presence or absence of a biological molecule by means of a reaction, comprising
(a) Establishing at least one hydrogel reaction chamber within a support substrate,
said hydrogel reaction chamber encapsulated within said support substrate with at least one substantially planar surface not in direct contact with said support substrate and available for administration of a sample capable of interrogation by means of a reaction undertaken within said hydrogel reaction chamber;
said support substrate hydrophobic and substantially solid at the temperature of non-operational handling or transport,
wherein at temperatures relevant for monitoring of reactions within said hydrogel reaction chambers, said support substrate becomes optically transparent at wavelengths relevant for the monitoring of said reactions undertaken in said hydrogel reaction chambers;
(b) Administering to said hydrogel reaction chamber a sample capable of interrogation by means of a reaction undertaken within said hydrogel reaction chamber;
(c) Initiating a reaction within said at least one hydrogel reaction chamber capable of producing an optical signal indicating the presence or absence of a biological molecule; and
(d) Detecting the presence or absence of said optical signal.

18. The method of claim 17 wherein said hydrophobic material is fluid at temperatures relevant for the monitoring of reaction undertaken in said hydrogel reaction chambers.

19. The method of claim 18 wherein said hydrophobic material is optically transparent and non-fluorescing at wavelengths of 390-420 nm.

20. The method of claim 19 wherein said hydrophobic material is optically transparent and non-fluorescent at a wavelength of 405 nm.

21. The method of claim 20 wherein said hydrophobic material is paraffin.

22. The method of claim 17 wherein the reaction undertaken in said hydrogel reaction chamber is selected from the group comprised of reverse transcription, polymerase chain reaction, isothermal polymerase chain reaction, immunostaining, immunohistochemistry, immunolabeling, in situ hybridization, and viral lysis of eukaryotic and prokaryotic cells.

23. The method of claim 17 wherein the sample capable of interrogation by means of a reaction undertaken within the hydrogel reaction chamber is a human clinical sample.

24. The method of claim 23 wherein the human clinical sample is a tissue sample suspended in at least water.

25. The method of claim 24 wherein the human clinical sample is selected from the group comprised of blood, macerated tissue, lymphatic fluid, genital swab, nasopharyngeal swab, buccal swab, skin swab, bone marrow, saliva, urine, and fecal matter.

26. A method of interrogating a sample for the presence or absence of a biological molecule by means of a reaction, comprising
(a) Establishing at least one hydrogel reaction chamber within a support substrate,
said hydrogel reaction chamber encapsulated within said support substrate with at least one substantially planar surface not in direct contact with said support substrate and available for administration of a sample capable of interrogation by means of a reaction undertaken within said hydrogel reaction chamber;
said support substrate hydrophobic and substantially solid at the temperature of non-operational handling or transport,
wherein said support substrate is optically transparent at wavelengths relevant for the monitoring of said reactions undertaken in said hydrogel reaction chambers;
(b) Administering to said hydrogel reaction chamber a sample capable of interrogation by means of a reaction undertaken within said hydrogel reaction chamber;
(c) Initiating a reaction within said at least one hydrogel reaction chamber capable of producing an optical signal indicating the presence or absence of a biological molecule; and
(d) Detecting the presence or absence of said optical signal.

27. The method of claim 26 wherein said hydrophobic material is fluid at temperatures relevant for the monitoring of reaction undertaken in said hydrogel reaction chambers.

28. The method of claim 27 wherein said hydrophobic material is optically transparent and non-fluorescing at wavelengths of 390-420 nm.

29. The method of claim 28 wherein said hydrophobic material is optically transparent and non-fluorescent at a wavelength of 405 nm.

30. The method of claim 29 wherein said hydrophobic material is paraffin.

31. The method of claim 30 wherein the reaction undertaken in said hydrogel reaction chamber is selected from the group comprised of reverse transcription, polymerase chain reaction, isothermal polymerase chain reaction, immunostaining, immunohistochemistry, immunolabeling, in situ hybridization, and viral lysis of eukaryotic and prokaryotic cells.

32. The method of claim 26 wherein the sample capable of interrogation by means of a reaction undertaken within the hydrogel reaction chamber is a human clinical sample.

33. The method of claim 32 wherein the human clinical sample is a tissue sample suspended in at least water.

34. The method of claim 33 wherein the human clinical sample is selected from the group comprised of blood, macerated tissue, lymphatic fluid, genital swab, nasopharyngeal swab, buccal swab, skin swab, bone marrow, saliva, urine, and fecal matter.

35. A method of interrogating a sample for the presence or absence of a biological molecule by means of a reaction, comprising
(a) Establishing at least one hydrogel reaction chamber containing a sample within a support substrate,
said hydrogel reaction chamber encapsulated within said support substrate with at most one surface of the hydrogel reaction chamber not in direct contact with said support substrate;
said support substrate hydrophobic and substantially solid at the temperature of non-operational handling or transport,
wherein said support substrate is optically transparent at wavelengths relevant for the monitoring of said reactions undertaken in said hydrogel reaction chambers;
(b) Initiating a reaction within said at least one hydrogel reaction chamber capable of producing an optical signal indicating the presence or absence of a biological molecule; and
(c) Detecting the presence or absence of said optical signal.

36. The method of claim 35 wherein said hydrophobic material is fluid at temperatures relevant for the monitoring of reaction undertaken in said hydrogel reaction chambers.

37. The method of claim 36 wherein said hydrophobic material is optically transparent and non-fluorescing at wavelengths of 390-420 nm.

38. The method of claim 37 wherein said hydrophobic material is optically transparent and non-fluorescent at a wavelength of 405 nm.

39. The method of claim 38 wherein said hydrophobic material is paraffin

40. The method of claim 35 wherein the reaction undertaken in said hydrogel reaction chamber is selected from the group comprised of reverse transcription, polymerase chain reaction, isothermal polymerase chain reaction, immunostaining, immunohistochemistry, immunolabeling, in situ hybridization, and viral lysis of eukaryotic and prokaryotic cells.

41. The method of claim 35 wherein the sample capable of interrogation by means of a reaction undertaken within the hydrogel reaction chamber is a human clinical sample.

42. The method of claim 41 wherein the human clinical sample is a tissue sample suspended in at least water.

43. The method of claim 42 wherein the human clinical sample is selected from the group comprised of blood, macerated tissue, lymphatic fluid, genital swab, nasopharyngeal swab, buccal swab, skin swab, bone marrow, saliva, urine, and fecal matter.

44. A support substrate for isolating a multiplicity of hydrogel reaction chambers encapsulated within said support substrate, with at most one surface of the hydrogel reaction chamber not in direct contact with said support substrate; said novel support substrate comprised of a hydrophobic material substantially solid at the temperature of non-operational handling or transport, wherein at temperatures relevant for monitoring of reactions within said hydrogel reaction chambers, said hydrophobic material is optically transparent at wavelengths relevant for the monitoring of said reactions undertaken in said hydrogel reaction chambers.

45. The support substrate of claim 44 wherein said hydrophobic material is fluid at temperatures relevant for the monitoring of reaction undertaken in said hydrogel reaction chambers.

46. The support substrate of claim 45 wherein said hydrophobic material is optically transparent and non-fluorescing at wavelengths of 390-420 nm.

47. The support substrate of claim 46 wherein said hydrophobic material is optically transparent and non-fluorescent at a wavelength of 405 nm.

48. The support substrate of claim 47 wherein said hydrophobic material is paraffin.

49. A support substrate for isolating a multiplicity of hydrogel reaction chambers encapsulated within said support substrate with at least one substantially planar surface not in direct contact with said support substrate and available for administration of a sample capable of interrogation by means of a reaction undertaken within said hydrogel reaction chamber; said novel support substrate comprised of a hydrophobic material substantially solid at the temperature of non-operational handling or transport, wherein at temperatures relevant for monitoring of reactions within said hydrogel reaction chambers, said hydrophobic material is optically transparent at wavelengths relevant for the monitoring of said reactions undertaken in said hydrogel reaction chambers.

50. The support substrate of claim 49 wherein said hydrophobic material is fluid at temperatures relevant for the monitoring of reaction undertaken in said hydrogel reaction chambers.

51. The support substrate of claim 50 wherein said hydrophobic material is optically transparent and non-fluorescing at wavelengths of 390-420 nm.

52. The support substrate of claim 51 wherein said hydrophobic material is optically transparent and non-fluorescent at a wavelength of 405 nm.
